(19) United States
(12) Patent Application Publication
(10) Pub. No.: US 2006/0159954 A1
(43) Pub. Date: Jul. 20, 2006

(54) Metal-complex compound and organic electroluminescence device using the compound

(75) Inventors: Masa-aki Haga, Tokyo (JP); Taro Tsubomura, Tokyo (JP); Fumio Okuda, Chiba (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Idemitsu Kosan Co., Ltd., Tokyo (JP)

(21) Appl. No.: 11/269,808

(22) Filed: Nov. 9, 2005

(30) Foreign Application Priority Data
Nov. 10, 2004 (JP) 2004-326093

(51) Int. Cl. H01L 51/54; H05B 33/14; C09K 11/06; C07C 213/02

(52) U.S. Cl. 428/690; 546/2; 428/917; 313/504; 257/E51.044

(57) ABSTRACT

A metal-complex compound which comprises a tridentate chelate ligand and a cyano group ligand having a specified partial structure. An organic electroluminescence device which comprises at least one organic thin film layer sandwiched between a pair of electrodes consisting of an anode and a cathode, wherein the organic thin film layer comprises the metal-complex compound, which emits light by applying an electric voltage between the pair of electrodes. The present invention provides an organic EL device which emits blue light of high purity and of short wavelength with an enhanced current efficiency.

### TECHNICAL FIELD

The present invention relates to a novel metal-complex compound and an organic electroluminescence device using the compound. Particularly, the present invention relates to an organic electroluminescence device (“electroluminescence” will be referred to as “EL”, hereinafter) which emits blue light of high purity and of short wavelength with an enhanced efficiency of light emission, and to a metal-complex compound realizing it.

### BACKGROUND ART

The organic EL devices have been expected to be applied to color wide screen image display devices replacing liquid crystal display devices, and have been intensively developed. Recently, although displays using the organic EL devices have now been used in practical applications, full-color image display devices using the same are still in the course of development because they lack in sufficient light emitting property. In order for improving the light emitting property, very high-efficiency green organic light-emitting devices based on electrophosphorescence employing ortho metalized iridium complex (fac-tris(2-phenylpyridine)iridium) as a phosphorus light emitting material for the organic EL device are proposed. (refer to Non-patent literatures 1 and 2 which will be described later)

Because the current organic EL devices employing the phosphorus photoluminescence are limited to emitting only green light, coverage as the color display devices is narrow. Therefore, it has been demanded to develop organic EL devices which emit light of different colors from green with improved light emission property. Regarding particularly with EL devices which emit blue light, those having an external quantum yield exceeding 5% is not reported yet. Accordingly, an improvement in the EL devices which emit blue light, if possible, enables the display devices to display full colors or white light resultantly advancing toward practical use of phosphorus light EL device greatly.

Currently, developments about a compound having an iridium atom as a phosphorus photoluminescence complex are actively carried out, and Compound A below is known as a material employable for an EL device which emits green light. On the other hand, Compound B below is known as a material for an EL device which emits blue light, however, the EL device employing Compound B is not practical in view points of both lifetime and efficiency of the device. Accordingly, it is necessary to develop another complex for EL devices which emit blue light, however, any material except Compound B has not been found yet now.

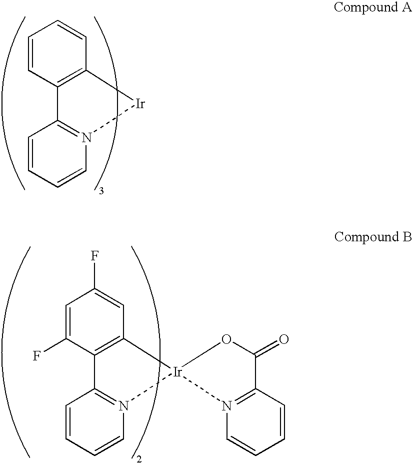

Although the above Compounds A and B are complexes having a bidentate chelate ligand, almost no complex having a tridentate chelate ligand similar to the above compounds is known except Compound C below. (refer to Non-patent literature 3 below)

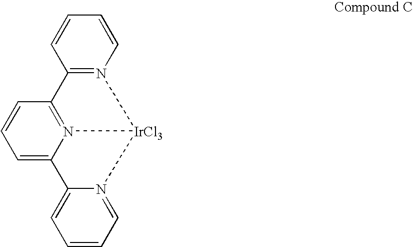

However, Compound C serves to emit reddish light having light emission wavelength of around 600 nm, without capability of serving to emit bluish light. Accordingly, a realization of a complex having a tridentate chelate ligand which serves to emit bluish light, if possible, has a possibility of new technology development.
Non-patent literature 1: D. F. O'Brien and M. A. Baldo et al.
   “Improved energy transfer in electrophosphorescent devices” Applied Physics letters Vol. 74 No. 3, pp 442-444, Jan. 18, 1999
Non-patent literature 2: M. A. Baldo et al.
   “Very high-efficiency green organic light-emitting devices based on electrophosphorescence” Applied Physics letters Vol. 75 No. 1, pp 4-6, Jul. 5, 1999
Non-patent literature 3: J-P. Collin et al.,
   J. Am. Chem. Soc., 121, 5009 (1999)

### DISCLOSURE OF THE INVENTION

The present invention has been made to overcome the above problems and has an object of providing an organic EL device which emits blue light of high purity and of short wavelength with an enhanced efficiency of light emission, and an object of providing a metal-complex compound realizing it.

The inventors clarified a novel structural factor for enabling to emit blue light that employing a metal-complex compound with partial structure having a tridentate chelate ligand and a cyano group ligand represented by a following general formula (I) enables to emit highly pure blue light of short wavelength and the present invention has been accomplished.

Namely, the present invention provides a metal-complex compound which comprises a tridentate chelate ligand and a cyano group ligand having a partial structure represented by a following general formula (I). 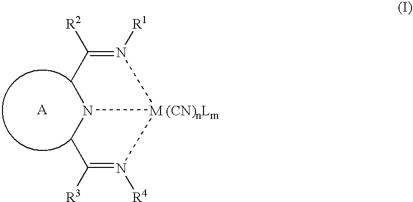
wherein M represents any one metal atom of Groups 7 to 12 in Periodic Table;
L represents a compound or an atomic group possessing any one atom of Groups 13 to 17 in Periodic Table;
A represents a heterocyclic group containing a nitrogen atom and having 2 to 20 carbon atoms which may have a substituent indicating that a circle enclosing the sign A shows a ring structure containing the nitrogen atom;
n represents an integer of 1 to 3, m represents an integer of 0 to 2;
R¹ to R⁴ each independently represents a hydrogen atom, a cyano group, a halogen atom, an alkyl group having 1 to 12 carbon atoms and further may have a substituent, an alkylamino group having 1 to 12 carbon atoms and further may have a substituent, an arylamino group, having 6 to 20 carbon atoms and further may have a substituent, an alkoxy group having 1 to 12 carbon atoms and further may have a substituent, an alkoxy halide group having 1 to 12 carbon atoms and further may have a substituent, an aryloxy group having 6 to 20 carbon atoms and further may have a substituent, an aromatic hydrocarbon group having 6 to 20 carbon atoms and further may have a substituent, a heterocyclic group having 3 to 20 carbon atoms and further may have a substituent, an alkyl halide group having 1 to 12 carbon atoms and further may have a substituent, an alkenyl group having 1 to 12 carbon atoms and further may have a substituent, an alkynyl group having 1 to 12 carbon atoms and further may have a substituent or a cycloalkyl group having 1 to 12 carbon atoms and further may have a substituent; while R¹ and R² or R³ and R⁴ may bond each other to form a ring structure.

Further, the present invention provides an organic EL device which comprises at least one organic thin film layer sandwiched between a pair of electrodes consisting of an anode and a cathode, wherein the organic thin film layer comprises the above metal-complex compound, which emits light by applying an electric voltage between the pair of electrodes.

The present invention provides an organic EL device which emits blue light of high purity and of short wavelength with an enhanced efficiency of light emission, and also provides a metal-complex compound realizing the EL device.

### BRIEF DESCRIPTION OF THE DRAWINGS

FIG. 1 is a diagram showing a light emission spectrum of Metal-Complex Compound 2 prepared in Synthesis Example 1 of the present invention.

### THE PREFERRED EMBODIMENT TO CARRY OUT THE INVENTION

The present invention provides a metal-complex compound having a partial structure represented by a following general formula (I):

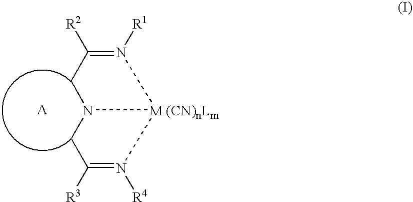

In the general formula (I), M represents any one metal atom of Groups 7 to 12 in Periodic Table, and examples include rhenium (Re) atom, iridium (Ir) atom, platinum (Pt) atom, rhodium (Rh) atom, osmium (Os), ruthenium (Ru) atom, cobalt (Co) atom, copper (Co) atom, zinc (Zn) atom, etc. Among those, any one metal atom of Group 9 in Periodic Table, Re or Pt is preferable and Ir, Re or Pt is particularly preferable.

In the general formula (I), n represents an integer of 1 to 3, m represents an integer of 0 to 2; n and m are determined dependently on a valence number of metal atom represented by the above M in order for maintaining the metal-complex compound neutral.

In the general formula (I), L represents a compound or an atomic group possessing any one atom of Groups 13 to 17 in Periodic Table. Examples of the atom of Groups 13 to 17 in Periodic Table possessed in the L include boron (B) atom, aluminum (Al) atom, carbon (C) atom, nitrogen (N) atom, oxygen (O) atom, silicon (Si) atom, phosphor (P) atom, sulfur (S) atom, germanium (Ge) atom, arsenic (As) atom, selenium (Se) atom, fluorine (F) atom, chlorine (Cl) atom, bromine (Br) atom, iodine (I) atom, etc; while P, Sb and N are preferable.

Examples of the above compound or the above atomic group represented by L is preferably at least one selected from a group consisting of PR, AsR, SbR, NR, OR, CO, a halogen atom, a heterocycle having 2 to 20 carbon atoms that may have a substituent, and a bidententate chelate ligand formed by combining those.

Examples of the halogen atom include fluorine atom, chlorine atom, bromine atom and iodine atom, etc.

Examples of the heterocycle having 2 to 20 carbon atoms include imidazole, benzimidazole, pyrrole, furan, thiophene, benzothiophene, oxadi azoline, diphenylanthracene, indoline, carbazole, pyridine, quinoline, isoquinoline, benzoquinone, pyrazoline, imidazolidine, piperidine, etc.

The above R represents a hydrogen atom, a cyano group, a halogen atom, an alkyl group having 1 to 12 carbon atoms and further may have a substituent, an alkylamino group having 1 to 12 carbon atoms and further may have a substituent, an arylamino group, having 6 to 20 carbon atoms and further may have a substituent, an alkoxy group having 1 to 12 carbon atoms and further may have a substituent, an alkoxy halide group having 1 to 12 carbon atoms and further may have a substituent, an aryloxy group having 6 to 20 carbon atoms and further may have a substituent, an aromatic hydrocarbon group having 6 to 20 carbon atoms and further may have a substituent, a heterocyclic group having 3 to 20 carbon atoms and further may have a substituent, an alkyl halide group having 1 to 12 carbon atoms and further may have a substituent, an alkenyl group having 1 to 12 carbon atoms and further may have a substituent, an alkynyl group having 1 to 12 carbon atoms and further may have a substituent or a cycloalkyl groups having 1 to 12 carbon atoms and further may have a substituent; and a number of R may be 2 or greater, plural of R may be the same with or different from each other.

Examples of the halogen atom include fluorine atom, chlorine atom, bromine atom and iodine atom, etc.

Examples of the alkyl group described above include methyl group, ethyl group, propyl group, isopropyl group, n-butyl group, s-butyl group, isobutyl group, t-butyl group, n-pentyl group, n-hexyl group, n-heptyl group, n-octyl group, etc.

Examples of the aromatic hydrocarbon group include moieties of benzene, naphthalene, anthracene, phenanthrene, pyrene, biphenyl, terphenyl, fluoranthene, etc.

Examples of the heterocyclic group include moieties of the examples of the foregoing heterocycle having 2 to 20 carbon atoms.

Examples of the alkylamino group include a group formed by substituting a hydrogen atom of the amino group with the above alkyl group.

Examples of the arylamino group include a group formed by substituting a hydrogen atom of the amino group with the aromatic hydrocarbon group.

The alkoxy group is expressed as —OY′, wherein Y′ represents the same as the foregoing examples about the above alkyl group,

Examples of the alkoxy halide group include a group formed by substituting a hydrogen atom of the alkoxy group with the above hologen atom.

The aryloxy group is expressed as —OY″, wherein Y″ represents the same as the foregoing examples about the above aromatic hydrocarbon group.

Examples of the alkyl halide group include a group formed by substituting a hydrogen atom of the alkyl group with the above halogen atom.

Examples of the alkenyl group include vinyl group, allyl group, 2-butenyl group, 3-pentenyl group, etc.

Examples of the alkynyl group include ethynyl group, methyl ethynyl group, etc.

Examples of the cycloalkyl group include cyclopropyl group, cyclobutyl group, cyclopentyl group, cyclohexyl group, etc.

Further, examples of the substituent for those groups include halogen atom, hydroxyl group, substituted or unsubstituted amino group, nitro group, cyano group, substituted or unsubstituted alkyl group, fluorination alkyl group, substituted or unsubstituted alkenyl group, a substituted or unsubstituted cycloalkyl group, substituted or unsubstituted alkoxyl group, substituted or unsubstituted heterocyclic group, substituted or unsubstituted arylalkyl group, a substituted or unsubstituted aryloxy group, substituted or unsubstituted alkoxycarbonyl group, carboxyl group, etc.

Further, it is also preferable for the compound or the atomic group represented by the foregoing L is any one expressed by following general formulae (1) to (6).

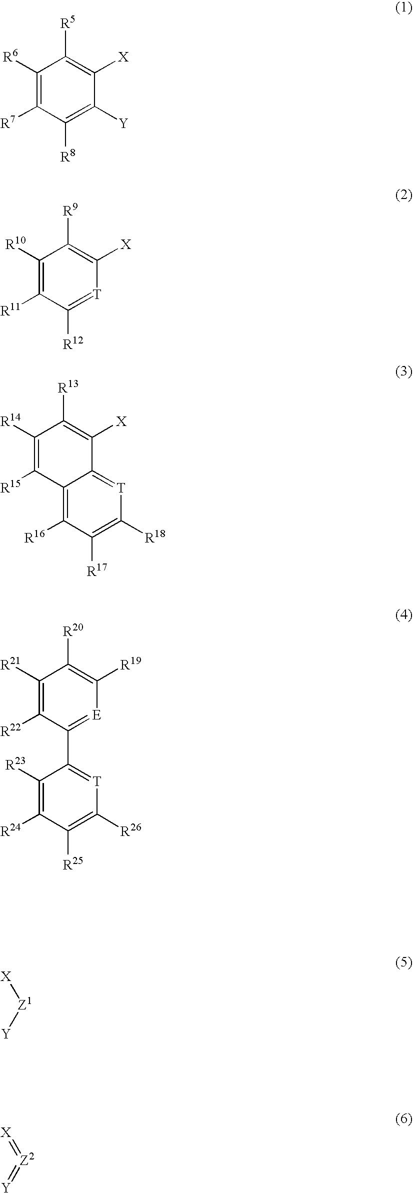

In the above general formulae (1) to (3), (5) and (6), X and Y each independently represents an oxygen (O) atom, a sulfur (S) atom, a nitrogen (N) atom, a phosphor (P) atom, an arsenic (As) atom or a antimony (Sb) atom.

In the above general formulae (2) to (4), T and E each independently represents a nitrogen (N) atom, a phosphor (P) atom, an arsenic (As) atom or an antimony (Sb) atom.

In the above general formula (5), Z¹ represents —(CH₂)_{q}—(q represents an integer of 1 to 3), —CH═CH— or —CH═C═CH—.

In the above general formula (6), Z² represents ═CR²⁷—CR²⁸═.

In the general formulae (1) to (6), R⁵ to R²⁸ each independently represents a hydrogen atom, a cyano group, a halogen atom, an alkyl group having 1 to 12 carbon atoms and further may have a substituent, an alkylamino group having 1 to 12 carbon atoms and further may have a substituent, an arylamino group, having 6 to 20 carbon atoms and further may have a substituent, an alkoxy group having 1 to 12 carbon atoms and further may have a substituent, an alkoxy halide group having 1 to 12 carbon atoms and further may have a substituent, an aryloxy group having 6 to 20 carbon atoms and further may have a substituent, an aromatic hydrocarbon group having 6 to 20 carbon atoms and further may have a substituent, a heterocyclic group having 3 to 20 carbon atoms and further may have a substituent, an alkyl halide group having 1 to 12 carbon atoms and further may have a substituent, an alkenyl group having 1 to 12 carbon atoms and further may have a substituent, an alkynyl group having 1 to 12 carbon atoms and further may have a substituent or a cycloalkyl groups having 1 to 12 carbon atoms and further may have a substituent; and specific examples of each groups and their substituents are the same as described about the foregoing R.

Further, an adjacent couple among R⁵ to R²⁸ may bond each other to form a ring structure. Examples of the ring structure include cycloalkane (for example, cyclopropane, cyclobutane, cyclopropane, cyclohexane, cycloheptane, etc.), aromatic hydrocarbon ring (for example, benzene, naphthalene, anthracene, phenanthrene, pyrene, biphenyl, terphenyl, fluoranthene, etc.) and heterocycle (for example, imidazole, benzimidazole, pyrrole, furan, thiophene, benzothiophene, oxadi azoline, diphenylanthracene, indoline, carbazole, pyridine, quinoline, isoquinolne, benzoquinone, pyrazoline, imidazoldine, piperidine, etc.).

Specific examples of the foregoing L include the following compounds: 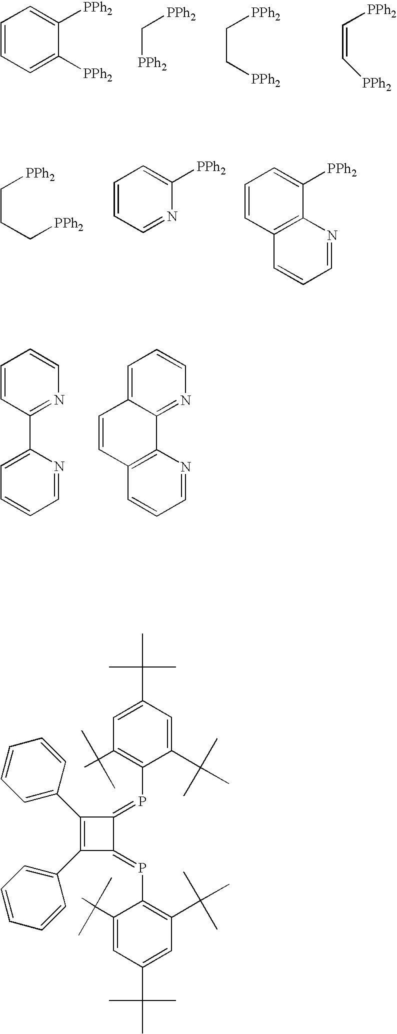
wherein Ph is a phenyl group.

In the general formula (I), A represents a heterocyclic group containing a nitrogen atom and having 2 to 20 carbon atoms which may have a substituent indicating that a circle enclosing the sign A shows a ring structure containing the nitrogen atom. Examples of A include imidazole, benzimidazole, pyrrole, indoline, carbazole, pyridine, quinoline, isoquinoline, pyrazoline, imidazolidine, piperidine, etc.; while pyridine is preferable.

Specific substituents of those compounds are the same as the foregoing description.

In the general formula (I), R¹ to R⁴ represents the same as described about the foregoing R⁵ to R²⁸, and specific examples of each group and their substituents are the same as described about the foregoing R.

Further, a couple of R¹ and R² or a couple of R³ and R⁴ may bond each other to form a ring structure. Examples of the ring structure are the same as described about the foregoing R⁵ to R²⁸.

In the general formula (I), the above tridentate chelate ligand is preferably any one of compounds shown by following general formula (7) or general formula (8).

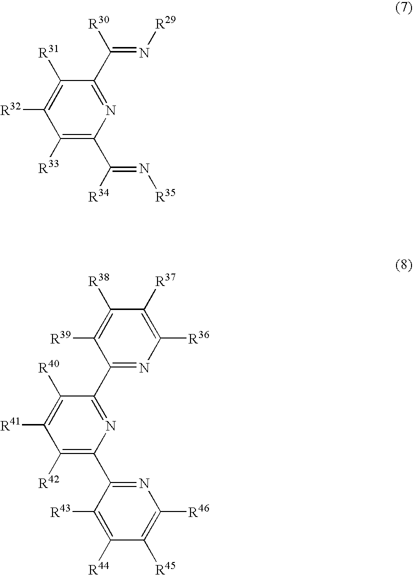

In the general formulae (7) and (8), R²⁹ to R³⁵ and R³⁶ to R⁴⁶ each independently represents the same as described about the foregoing R¹ to R⁴, and specific examples of each group and their substituent are the same as described about the foregoing R.

Further, an adjacent couple among R²⁹ to R³⁵ and R³⁶ to R⁴⁶ may bond each other to form a ring structure, while examples of the ring structure being the same as explained about the foregoing R⁵ to R²⁸.

Furthermore, it is preferable that the tridentate chelate ligand is any one of following compounds:

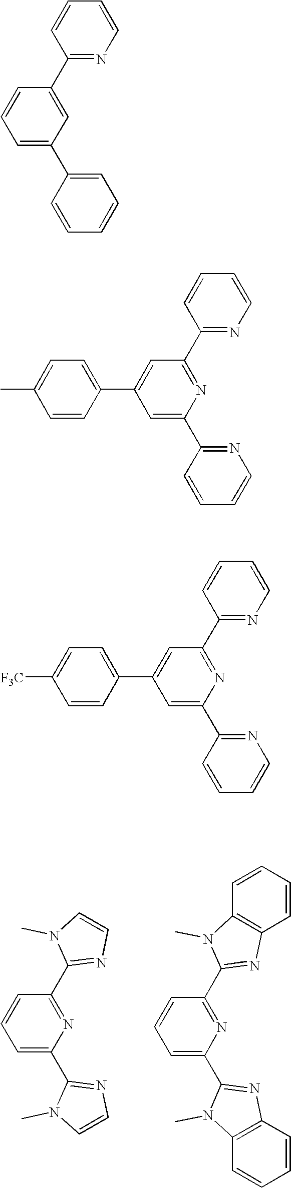

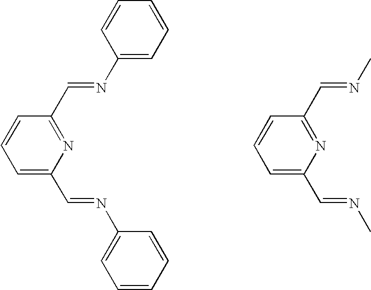

It is preferable that the metal-complex compound of the present invention is expressed by following general formula (I-1) or general formula (I-2).

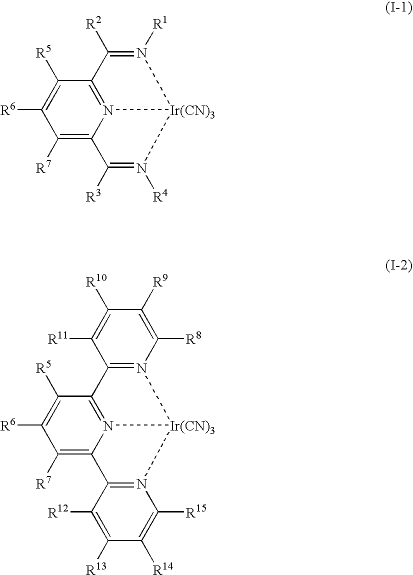

In the general formulae (I-1) and (I-2), R²⁹ to R³⁵ and R³⁶ to R⁴⁶ each independently represent the same as the foregoing description about themselves.

Specific examples of the metal-complex compound of the present invention are as follows, however, the present invention is not limited to these typical compounds.

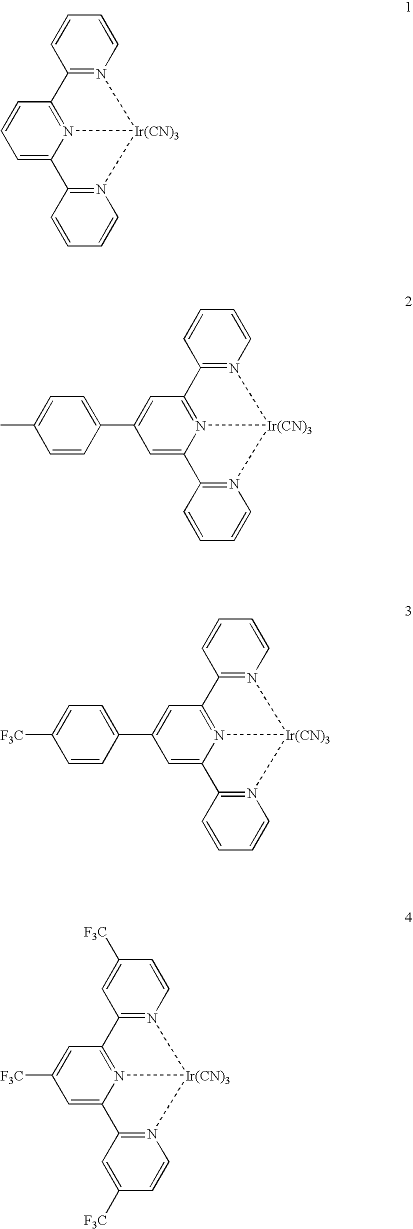

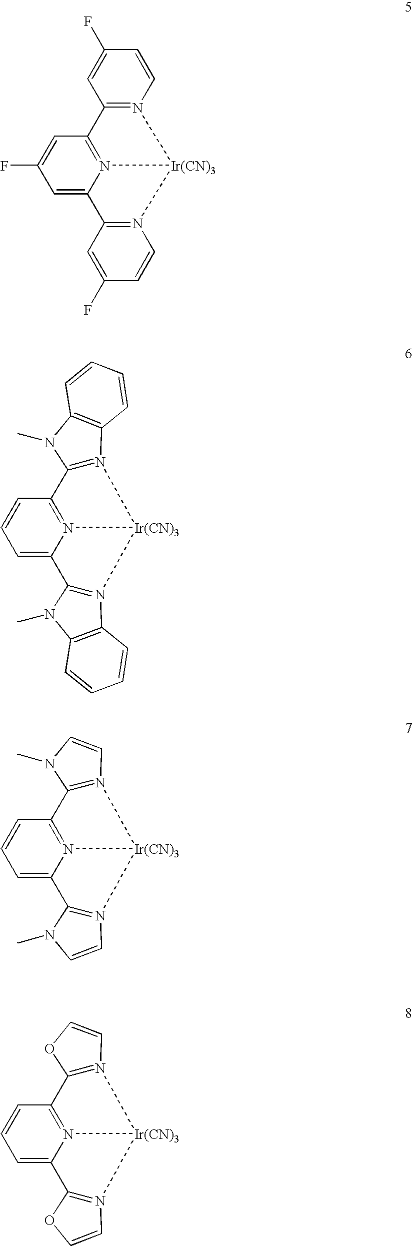

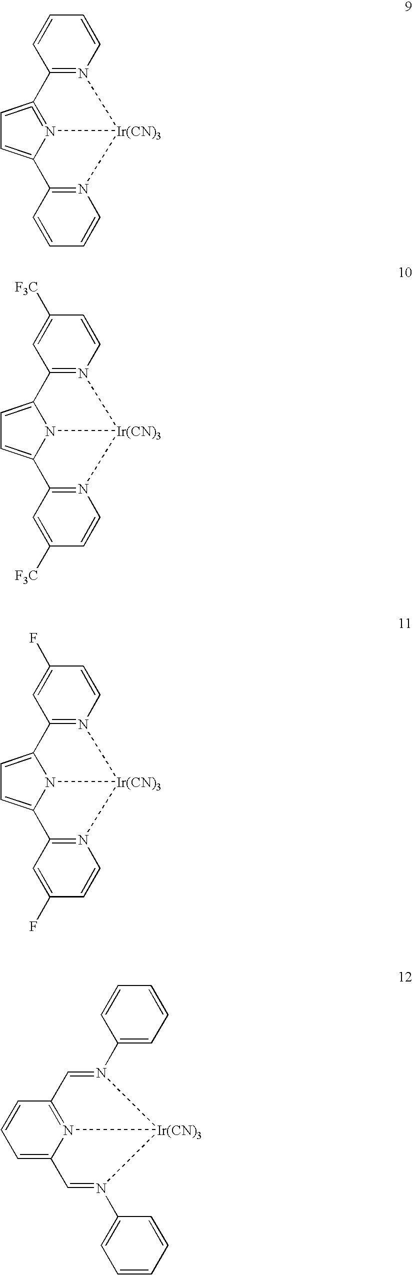

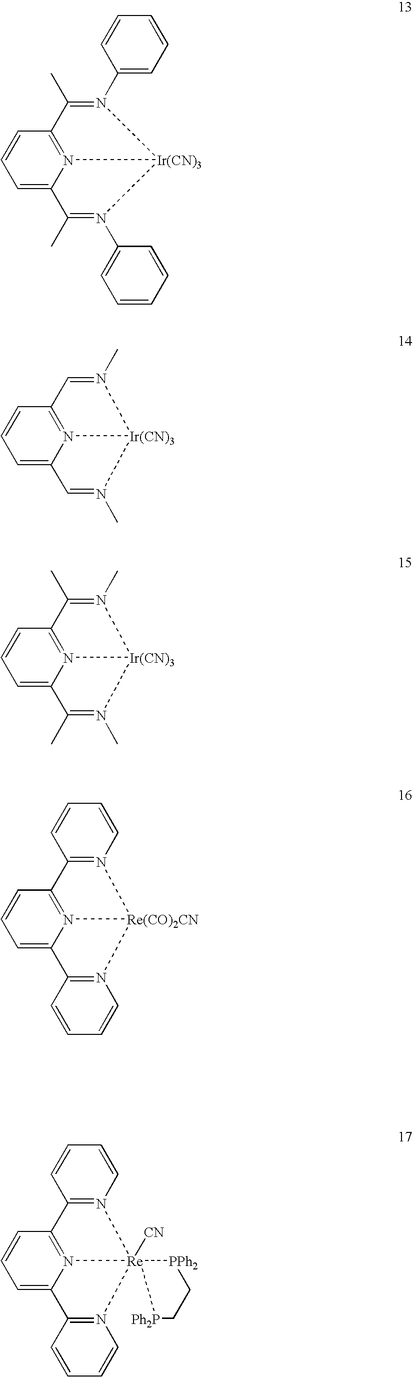

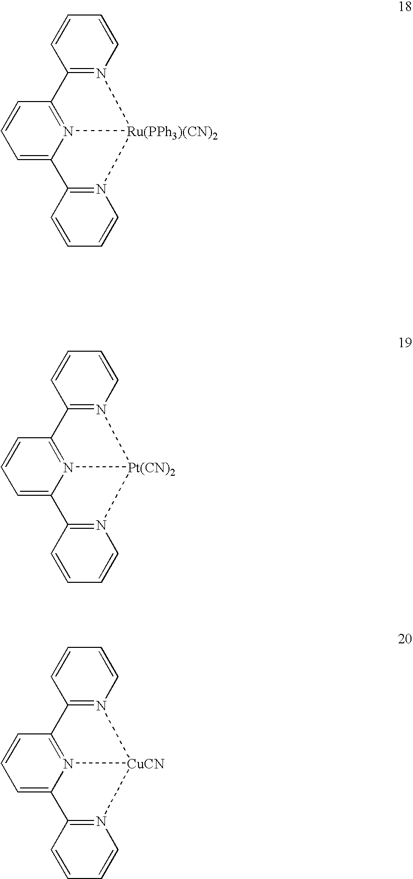

The present invention provides an organic EL device which comprises at least one organic thin film layer sandwiched between a pair of electrodes consisting of an anode and a cathode, wherein the organic thin film layer comprises the foregoing metal-complex compound, which emits light by applying an electric voltage between the pair of electrodes.

It is preferable for the organic EL device of the present invention that the light emitting layer comprises the metal-complex compound of the present invention, and that it comprises the metal-complex compound of the present invention in an amount of 1 to 30% by weight of total weight of the light emitting layer.

Further, the light emitting layer is usually formed to a thin film by means of vapor deposition process or coating process, however, it is preferable that the layer comprising the metal-complex compound of the present invention is formed into film by coating process because it simplifies the production process.

The organic EL device of the present invention is fabricated by sandwiching at least one organic layer between a pair of electrodes and examples of the construction include (i) an anode/a light emitting layer/a cathode; (ii) an anode/a hole injecting or a hole transporting layer/a light emitting layer/an electron injecting or an electron transporting layer/a cathode; (iii) an anode/a hole injecting or a hole transporting layer/a light emitting layer/an electron injecting or an electron transporting layer/a cathode; and (iV) an anode/a light emitting layer/an electron injecting or an electron transporting layer/a cathode.

The metal-complex compound in the present invention may be used in any of the foregoing organic layer, or may be doped into other hole transporting materials, light emitting materials and electron transporting materials. The process for forming the layers in the organic EL device of the present invention is not particularly limited. Except the vapor deposition process, after dissolving the light emitting composition of the present invention or after dissolving the compound forming the composition, the resultant solution may be formed into a light emitting medium or a light emitting layer by means of various wet processes. Namely, they may be formed in accordance with a conventional coating process such as the dipping process, the spin coating process, the casting process, the bar coating process and the roller coating process or with an ink-jet process. The thickness of each layer in the organic thin film layer in the organic EL device of the present invention is not particularly limited. In general, an excessively thin layer tends to have defects such as pin holes, and an excessively thick layer requires a high applied voltage resulting in decreasing the efficiency. Therefore, a thickness within the range of several nanometers to 1 μm is preferable.

Examples of the solvent used for preparing light emitting solution for the light emitting layer include, halogen-based hydrocarbon solvent such as dichloro-methane, dichloroethane, chloroform, tetrachloromethane, tetrachloro ethane, trichloroethane, chlorobenzene, dichlorobenzene, chlorotoluene, etc.; ether-based solvent such as dibutyl ether, tetrahydrofuran, dioxane, anisole, etc.; alcohol-based solvent such as methanol, ethanol, propanol, butanol, pentanol, hexanol, cyclohexanol, methyl cellosolve, ethylcellosolve, ethylene glycol, etc.; hydrocarbon-based solvent such as benzene, toluene, xylene, ethyl benzene, hexane, octane, decane, etc.; ester-based solvent such as ethyl acetate, butyl acetate, amyl acetate, etc. Among those, halogen-based hydrocarbon solvent, hydrocarbon-based solvent and ether-based solvent are preferable. Further, the solvent may be used alone, or in combination of two or more kind thereof. Additionally, the employable solvent is not limited to the above examples. Still further, a dopant may be optionally dissolved in advance, into the solution for the light emitting layer.

Electron injecting or transporting material employed for the present invention is not particularly specified and any compound usually employed as electron injecting or transporting material may be employable. Examples include oxadiazole derivatives such as 2-(4-biphenyly)-5-(4-t-butylphenyl)-1,3,4-oxadiazole, bis {2-(4-t-butylphenyl)-1,3,4-oxadiazole}-m-phenylene, triazole derivatives and quinolinol-based metal-complex. As an inorganic compound for an electron injecting or transporting layer it is preferable to employ an insulating material or a semiconductor.

The electron injecting or transporting layer effectively prevents leak in the electric current and improves the electron injecting capability. It is preferable that at least one metal compound selected from the group consisting of alkali metal chalcogenides, alkaline earth metal chalcogenides, alkali metal halides and alkaline earth metal halides is used as the insulating material. It is preferable that the electron injecting or transporting layer is constituted with the above alkali metal chalcogenide since the electron injecting property can be improved.

Preferable examples of the alkali metal chalcogenide include Li₂O, LiO, Na₂S and Na₂Se. Preferable examples of the alkaline earth metal chalcogenide include CaO, BaO, SrO, BeO, BaS and CaSe. Preferable examples of the alkali metal halide include LiF, NaF, KF, LiCl, KCl and NaCl. Preferable examples of the alkaline earth metal halide include fluorides such as CaF₂, BaF₂, SrF₂, MgF₂ and BeF₂ and halides other than the fluorides.

Examples of the semiconductor constituting the electron injecting or transporting layer include oxides, nitrides and nitriding oxides containing at least one element selected from Ba, Ca, Sr, Yb, Al, Ga, In, Li, Na, Cd, Mg, Si, Ta, Sb and Zn, which are used singly or in combination of two or more. It is preferable that the inorganic compound constituting the electron injecting or transporting layer is in the form of a fine crystalline or amorphous insulating thin film. When the electron injecting or transporting layer is constituted with the above insulating thin film, a more uniform thin film can be formed and defective pixels such as dark spots can be decreased. Examples of the inorganic compound include the alkali metal chalcogenides, the alkaline earth metal chalcogenides, the alkali metal halides and the alkaline earth metal halides which are described above.

In the present invention, a reductive dopant with a work function of 2.9 eV or smaller may be added in the electron injecting or transporting layer. The reductive dopant used in the present invention is defined as a substance which reduces the electron transporting compound. Accordingly, various compounds having a reductive capability are employable and examples include at least one compound selected from alkali metals, alkali metallic complexes, alkali metal compounds, alkaline earth metals, alkaline earth metallic complexes, alkaline earth metal compounds, rare earth metals, rare earth metallic complexes and rare earth metal compounds.

Examples of the preferable reductive dopant include at least one alkali metal selected from a group consisting of Li (the work function: 2.93 ev), Na (the work function: 2.36 eV), K (the work function: 2.28 eV), Rb (the work function: 2.16 eV) and Cs (the work function: 1.95 eV) or at least one alkaline earth metals selected from a group consisting of Ca (the work function: 2.9 eV), Sr (the work function: 2.0 to 2.5 eV) and Ba (the work function: 2.52 eV); whose work function of 2.9 eV or smaller is particularly preferable. Among those, more preferable reductive dopants include at least one kind selected from the group consisting of K, Rb and Cs, the latter Rb or Cs being farther more preferable and the last Cs being the most preferable. Those alkaline metals have particularly high reducing capability, and only an addition of relatively small amount of them into an electron injection zone enables to expect both improvement of luminance and lifetime extension of the organic EL device.

Further, with regard to the reductive dopant with work function of 2.9 eV or smaller, a combination of two or more kinds of the alkali metal is also preferable, and particularly, combinations containing Cs, for example, combinations of Cs and Na, Cs and K, Cs and Rb, Cs and Na and K are preferable. Containing Cs in combination enables to reveal reducing capability effectively, and the addition into the electron injection zone expects both improvement of luminance and lifetime extension of the organic EL device.

The anode in the organic EL device covers a role of injecting holes into a hole injecting or transporting layer or into a light emitting layer, and it is effective that the anode has a work function of 4.5 eV or greater. Specific examples of the material for the anode include indium tin oxide (ITO) alloy, tin oxide (NESA), gold, silver, platinum, copper, etc. With regard to the cathode, its material preferably has a small work function with the aim of injecting electrons into an electron injecting or transporting layer or into a light emitting layer. Further in the organic EL device, a hole injecting (transporting) layer may be disposed over the anode. Various organic compounds and polymers usually used for the organic EL device, for example, which are described in Japanese Unexamined Patent Application Laid-Open Nos. Shou 63-295695 and Hei 2-191694 may be employed as the hole injecting or transporting layer. Examples include aromatic tertiary amine, hydrazone derivative, carbazole derivatives, triazole derivatives, imidazole derivatives or polyvinylcarbazole, polyethylendihydroxythiophene poly sulfonic acid (PEDOT/PSS), etc.

Although materials for the cathode of the organic EL device are not particularly specified, examples include indium, aluminum, magnesium, magnesium-indium alloy, magnesium-aluminum alloy, aluminum-lithium alloy, aluminum-scandium-lithium alloy, magnesium-silver alloy, etc.

### EXAMPLE

The present invention shall be explained below in further details with reference to examples, but the present invention shall by no means be restricted by the following examples.

### Synthesis Example 1 (Synthesis of Metal-Complex Compound 2)

The route for synthesis of the above Metal-Complex Compound 2 is illustrated as follows:

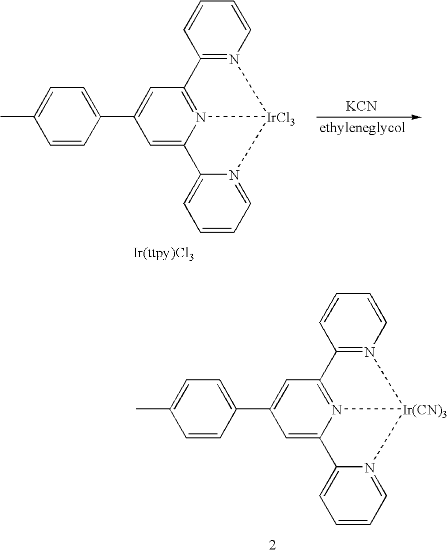

Placing 0.1 g (0.16 mmol) of Ir(ttpy)Cl₃, 15 milliliter of ethylene glycol and 0.064 g (0.96 mmol) of KCL into an egg plant type flask having a capacity of 100 milliliter, the resultant solution was exposed to microwave irradiation by means of 650 W microwave irradiation equipment (ZMW-007 type; produced by Shikoku Instrumentation Co., Ltd) intermittently 4 times for 3 minutes while stirring under heating. After the resultant solution was cooled to room temperature by leaving it standing, 100 milliliter of pure water was added, followed by stirring for 30 minutes. Afterwards, a supernatant solution was removed with centrifugal separation manipulation, and a precipitate was collected by filtration. The precipitate was washed with the use of chloroform and diethylether, followed by drying, and as a result, 0.05 g of Metal-Complex Compound 2 as yellow powders was obtained (yield: 58%). The yellow powders were identified as the aimed compound from the result in accordance with Field Desorption Mass Spectrum (FD-MS) measurement. The result of the measurement in accordance with FD-MS is shown as the following:
FD-MS: calcd for IrC₂₅H₁₇N₆=593, found, m/z=593 (100)

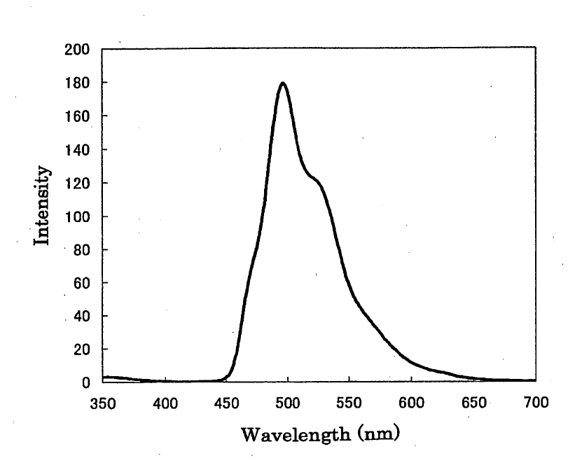

It was confirmed in accordance with a measurement of light emission spectrum about Metal-Complex Compound 2 obtained, that λ max=493 nm (excitation wavelength: 475 nm). The results are shown in FIG. 1.

As compared with the above result, the light emission spectrum about Ir(ttpy)Cl₃ employed as the material is known as λmax=610 nm. From the results, it is verified that the metal-complex compound having a tridentate ligand and further introducing a cyano group has an effect of extraordinarily shortening the wavelength of light emission.

### Example 1 (Fabrication of Organic EL Device)

A glass substrate (manufactured by GEOMATEC Company) of 25 mm×75 mm×1.1 mm thickness having an ITO transparent electrode was cleaned by application of ultrasonic wave in isopropyl alcohol for 5 minutes and then by exposure to ozone generated by ultraviolet light for 30 minutes. On the substrate, a film of polyethylene dihydroxy thiophene (PEDOT) for the use of the hole injecting layer with film thickness of 100 nm was formed in accordance with a spin coating process and then, a chloroform solution having a concentration of 0.5% by weight prepared by mixing Metal-Complex Compound 2 synthesized in Synthesis Example 1 in an amount of 7% by weight to Host Material H below and dissolved into a chloroform solvent made by bubbling nitrogen gas for 15 minutes under the same atmosphere was applied over PEDOT by means of a spin coating process to form a film. The coated film worked as a light emitting layer. The film thickness was 50 nm. On the film formed above, a film of BAlq below having a thickness 25 nm was formed. The formed film of BAlq worked as the hole barrier layer. On the film formed above, a film of Alq having a thickness 5 nm was formed. The film of Alq worked as the electron injecting layer. Subsequently, lithium fluoride was deposited up to 0.1 nm in thickness and then, aluminum was deposited up to 150 nm in thickness. The Al/LiF worked as a cathode. An organic EL device was fabricated in the manner described above. The device fabricated above was sealed and examined by feeding electric current. Bluish green light was emitted at a luminance of 100 cd/m² under a voltage of 7.6 V and a current density of 0.74 mA/cm². The CIE chromaticity coordinates were (0.17, 0.28), and the current efficiency was 13.5 cd/A.

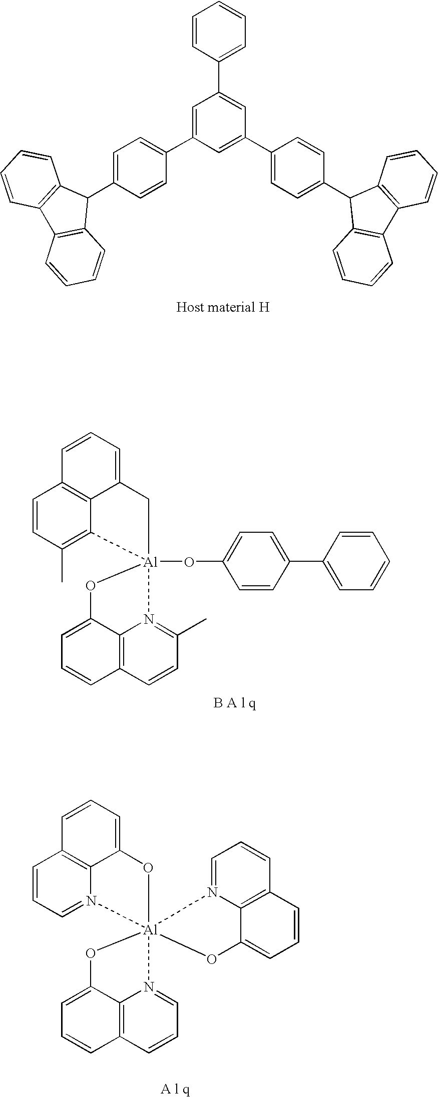

### Comparative Example 1

An organic EL device was fabricated similarly as Example 1 except that Metal-complex Compound D1 below described in publicly known literature Inorg. Chem., 6513 (2004) was used instead of Metal-Complex Compound 2.

The device fabricated above was sealed and examined by feeding electric current. Orange light was emitted at a luminance of 101 cd/m² under a voltage of 8.8 V and a current density of 0.68 mA/cm². The CIE chromaticity coordinates were (0.51, 0.48), and the current efficiency was 6.5 cd/A.

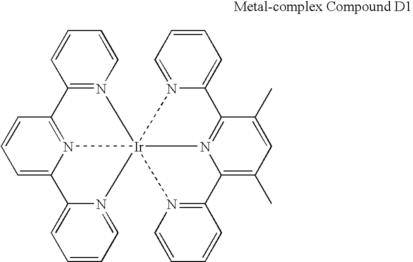

### Comparative Example 2

An organic EL device was fabricated similarly as Example 1 except that Ir(ttpy)Cl₃ above was used instead of Metal-complex Compound 2.

The device fabricated above was sealed and examined by feeding electric current. Red light was emitted at a luminance of 98 cd/m² under a voltage of 22.4 V and a current density of 8.48 mA/cm². The CIE chromaticity coordinates were (0.66, 0.39), and the current efficiency was 1.2 cd/A.

As the foregoing description, despite employing the same central metal and the tridentate chelate ligand, a case in the present invention where a metal-complex compound with the optimized ligand structure reduces the wavelength of light emission shorter, and provides an organic EL device of an enhanced current efficiency.

### INDUSTRIAL APPLICABILITY

As described above in detail, the present invention provides an organic EL device which emits blue light of high purity and of short wavelength with an enhanced current efficiency. Accordingly, the present invention is applicable for a field such as various display devices, display panels, backlights, illuminating light sources, beacon lights, signboards, and interior designs, particularly suitable as display device for color displays.

What is claimed is:

1. A metal-complex compound which comprises a tridentate chelate ligand and a cyano group ligand having a partial structure represented by a following general formula (I): 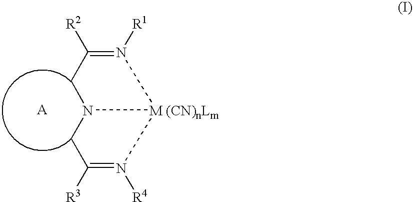
wherein M represents any one metal atom of Groups 7 to 12 in Periodic Table;
L represents a compound or an atomic group possessing any one atom of Groups 13 to 17 in Periodic Table;
A represents a heterocyclic group containing a nitrogen atom and having 2 to 20 carbon atoms which may have a substituent indicating that a circle enclosing the sign A shows a ring structure containing the nitrogen atom;
n represents an integer of 1 to 3, m represents an integer of 0 to 2;
R¹ to R⁴ each independently represents a hydrogen atom, a cyano group, a halogen atom, an alkyl group having 1 to 12 carbon atoms and further may have a substituent, an alkylamino group having 1 to 12 carbon atoms and further may have a substituent, an arylamino group, having 6 to 20 carbon atoms and further may have a substituent, an alkoxy group having 1 to 12 carbon atoms and further may have a substituent, an alkoxy halide group having 1 to 12 carbon atoms and further may have a substituent, an aryloxy group having 6 to 20 carbon atoms and further may have a substituent, an aromatic hydrocarbon group having 6 to 20 carbon atoms and further may have a substituent, a heterocyclic group having 3 to 20 carbon atoms and further may have a substituent, an alkyl halide group having 1 to 12 carbon atoms and further may have a substituent, an alkenyl group having 1 to 12 carbon atoms and further may have a substituent, an alkynyl group having 1 to 12 carbon atoms and further may have a substituent or a cycloalkyl groups having 1 to 12 carbon atoms and further may have a substituent; while R¹ and R² or R³ and R⁴ may bond each other to form a ring structure.

2. The metal-complex compound according to claim 1, wherein said L is at least one kind selected from a group consisting of PR, AsR, SbR, NR, OR wherein R represents a hydrogen atom, a cyano group, a halogen atom, an alkyl group having 1 to 12 carbon atoms and further may have a substituent, an alkylamino group having 1 to 12 carbon atoms and further may have a substituent, an arylamino group, having 6 to 20 carbon atoms and further may have a substituent, an alkoxy group having 1 to 12 carbon atoms and further may have a substituent, an alkoxy halide group having 1 to 12 carbon atoms and further may have a substituent, an aryloxy group having 6 to 20 carbon atoms and further may have a substituent, an aromatic hydrocarbon group having 6 to 20 carbon atoms and further may have a substituent, a heterocyclic group having 3 to 20 carbon atoms and further may have a substituent, an alkyl halide group having 1 to 12 carbon atoms and further may have a substituent, an alkenyl group having 1 to 12 carbon atoms and further may have a substituent, an alkynyl group having 1 to 12 carbon atoms and further may have a substituent or a cycloalkyl groups having 1 to 12 carbon atoms and further may have a substituent; and a number of R may be 2 or greater, plural of R may be the same with or different from each other; and
CO, a halogen atom, a heterocyclic group having 3 to 20 carbon atoms and further may have a substituent, and a bidententate chelate ligand.

3. The metal-complex compound according to claim 1, wherein said L is a compound expressed by any one of general formulae (1) to (6) below. 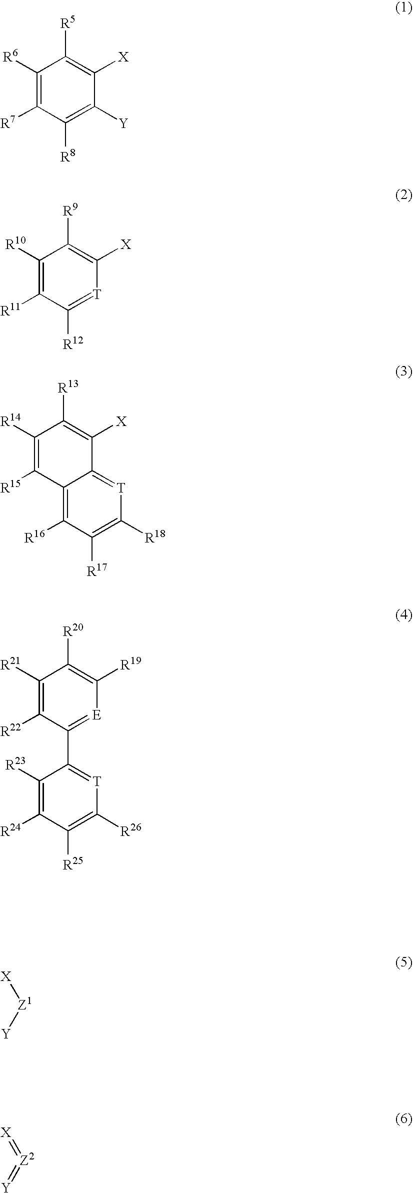
wherein X and Y each independently represents an oxygen (O) atom, a sulfur (S) atom, a nitrogen (N) atom, a phosphor (P) atom, an arsenic (As) atom or an antimony (Sb) atom;
T and E each independently represents a nitrogen (N) atom, a phosphor (P) atom, an arsenic (As) atom or an antimony (Sb) atom;
Z¹ represents —(CH₂)_{q}—, —CH═CH— or —CH═C═CH—; wherein q represents an integer of 1 to 3;
Z² represents ═CR²⁷—CR²⁸═;
R⁵ to R²⁸ each independently represents a hydrogen atom, a cyano group, a halogen atom, an alkyl group having 1 to 12 carbon atoms and further may have a substituent, an alkylamino group having 1 to 12 carbon atoms and further may have a substituent, an arylamino group, having 6 to 20 carbon atoms and further may have a substituent, an alkoxy group having 1 to 12 carbon atoms and further may have a substituent, an alkoxy halide group having 1 to 12 carbon atoms and further may have a substituent, an aryloxy group having 6 to 20 carbon atoms and further may have a substituent, an aromatic hydrocarbon group having 6 to 20 carbon atoms and further may have a substituent, a heterocyclic group having 3 to 20 carbon atoms and further may have a substituent, an alkyl halide group having 1 to 12 carbon atoms and further may have a substituent, an alkenyl group having 1 to 12 carbon atoms and further may have a substituent, an alkynyl group having 1 to 12 carbon atoms and further may have a substituent or a cycloalkyl groups having 1 to 12 carbon atoms and further may have a substituent; and an adjacent couple among R⁵ to R²⁸ may bond each other to form a ring structure.

4. The metal-complex compound according to claim 1, wherein said M in the general formula (I) is any one of metal atom of Group 9 in Periodic Table.

5. The metal-complex compound according to claim 1, wherein said M in the general formula (I) is a rhenium (Re) atom, an iridium (Ir) atom, a platinum (Pt) atom, a rhodium (Rd) atom, an osmium (Os) atom or a ruthenium (Ru) atom.

6. The metal-complex compound according to claim 1, wherein said M in the general formula (I) is an iridium (Ir) atom or a platinum (Pt) atom.

7. The metal-complex compound according to claim 1, wherein said M in the general formula (I) is a rhenium (Re) atom.

8. The metal-complex compound according to claim 1, wherein said tridentate chelate ligand in the general formula (I) is a compound expressed by following general formula (7): 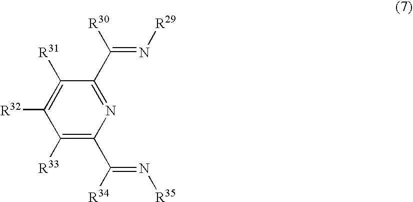
wherein R²⁹ to R³⁵ each independently represents the same as described about the above R¹ to R⁴, and an adjacent couple among R²⁹ to R³⁵ may bond each other to form a ring structure.

9. The metal-complex compound according to claim 1, wherein said tridentate chelate ligand in the general formula (I) is a compound expressed by following general formula (8): 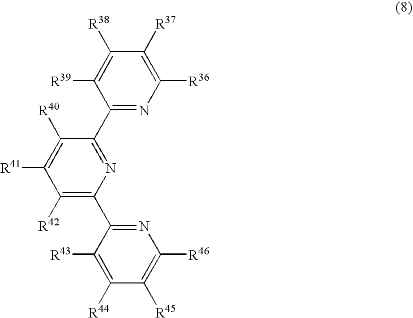
wherein R³⁶ to R⁴⁶ each independently represents the same as described about the above R¹ to R⁴, and an adjacent couple among R³⁶ to R⁴⁶ may bond each other to form a ring structure.

10. The metal-complex compound according to claim 1, which is expressed by a following general formula (I-1): 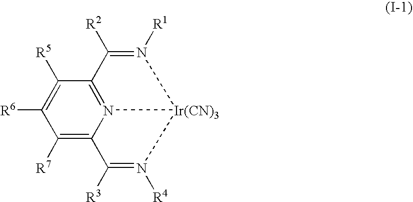
wherein R²⁹ to R³⁵ each independently represents the same as described about the above R¹ to R⁴, and an adjacent couple among R²⁹ to R³⁵ may bond each other to form a ring structure.

11. The metal-complex compound according to claim 1, which is expressed by a following general formula (I-2): 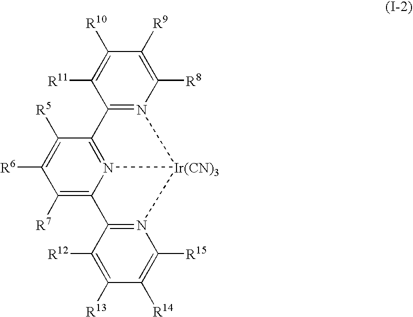
wherein R³⁶ to R⁴⁶ each independently represents the same as described about the above R¹ to R⁴, and an adjacent couple among R²⁹ to R³⁵ may bond each other to form a ring structure.

12. An organic electroluminescence device which comprises at least one organic thin film layer sandwiched between a pair of electrodes consisting of an anode and a cathode, wherein the organic thin film layer comprises the metal-complex compound according to any one of claims 1 to 11, which emits light by applying an electric voltage between the pair of electrodes.

13. The organic electroluminescence device according to claim 12, wherein said metal-complex compound is contained in a light emitting layer.

14. The organic electroluminescence device according to claim 12 or claim 13, which emits bluish light.

15. The organic electroluminescence device according to any one of claims 12 to 14, wherein said organic thin film layer comprising the metal-complex compound is formed by coating process.
